(19) United States
(12) Patent Application Publication
(10) Pub. No.: US 2022/0041984 A1
(43) Pub. Date: Feb. 10, 2022

(54) MOBILIZED PERIPHERAL BLOOD AS A SOURCE OF MODIFIED IMMUNE CELLS

(71) Applicant: THE TRUSTEES OF THE UNIVERSITY OF PENNSYLVANIA, Philadelphia, PA (US)

(72) Inventor: Saar GILL, Philadelphia, PA (US)

(21) Appl. No.: 17/189,970

(22) Filed: Mar. 2, 2021

Related U.S. Application Data
(60) Provisional application No. 62/983,968, filed on Mar. 2, 2020.

(51) Int. Cl. C12N 5/0783; C07K 14/725; C07K 16/30; A61K 35/17

(52) U.S. Cl. CPC C12N 5/0636; C07K 14/7051; C07K 16/30; C12N 2506/11; C07K 2319/02; C07K 2319/03; A61K 35/17

(57) ABSTRACT

The present invention relates to compositions and methods of generating sources of immune cells and/or stem cells for cell therapy. One aspect of the invention includes a method of generating an immune cell to be modified into an immune cell comprising a chimeric antigen receptor (CAR). Another aspect of the invention includes a method of generating cells, such as immune cells and/or stem cells, for autologous or allogeneic cell therapy. Also included are methods and pharmaceutical compositions comprising the cells for adoptive therapy and treating a condition, such as an autoimmune disease or cancer.

### CROSS-REFERENCE TO RELATED APPLICATIONS

This application claims priority to U.S. Provisional Application No. 62/983,968 filed Mar. 2, 2020, which is herein incorporated by reference in its entirety.

### STATEMENT REGARDING FEDERALLY SPONSORED RESEARCH OR DEVELOPMENT

This invention was made with government support under grant no. 5P01CA214278-02 awarded by the National Institute of Health. The government has certain rights in the invention.

### BACKGROUND OF THE INVENTION

Currently, T cells for genetic engineering (e.g. CART cells) are sourced from peripheral blood mononuclear cells (PBMC) circulating in the blood of patients or healthy donors, where the patient or donor undergoes a large-volume collection known as apheresis. In this procedure, 3-5 blood volumes (e.g. 15-25 liters) of blood are circulated through a machine that draws off PBMC and returns red blood cells, plasma and platelets to the donor.

A need exists for alternative sources for T cells to be used for genetic engineering (e.g., for manufacturing CART cells), where the source yields T cells of improved quality or function. A need also exists for sources of T cells and other cell types to be used for cell therapy, where efficiencies in cell collection are improved, and cells are obtained at lower costs and at less risk to patients or donors. The present invention satisfies these needs.

### SUMMARY OF THE INVENTION

In one aspect, the present disclosure provides a method of generating a T cell comprising a chimeric antigen receptor (CAR) from mobilized peripheral blood, the method comprising administering to a subject an agent that induces migration of stem cells from the subject's bone marrow to the subject's peripheral blood and introducing a CAR into a T cell from the mobilized peripheral blood obtained from the subject.

In another aspect, the present disclosure provides a method of generating a T cell comprising a chimeric antigen receptor (CAR) from mobilized peripheral blood, the method comprising

administering to a subject an agent that induces migration of stem cells from the subject's bone marrow to the subject's peripheral blood, thereby generating mobilized peripheral blood in the subject, and

introducing a CAR into a T cell obtained from peripheral blood mononuclear cells (PBMC) in the mobilized peripheral blood obtained from the subject.

In aspects, the present disclosure provides a method of generating stem cells and T cells, the T cells comprising a chimeric antigen receptor (CAR), from a single source, the method comprising

administering to a subject an agent that induces migration of stem cells from the subject's bone marrow to the subject's peripheral blood, thereby generating mobilized peripheral blood in the subject,

introducing a CAR into a T cell obtained from a mobilized peripheral blood obtained from the subject, and

obtaining a stem cell from the same mobilized peripheral blood sample obtained from the subject.

In other aspects, the present disclosure provides a method of generating a T cell comprising a chimeric antigen receptor (CAR), the method comprising

introducing a nucleic acid encoding a chimeric antigen receptor (CAR) into a T cell,

wherein the T cell is obtained from mobilized peripheral blood from a subject, wherein the mobilized peripheral blood is generated by administering to the subject an agent that induces migration of stem cells from the subject's bone marrow into the subject's peripheral blood.

In one aspect, the present disclosure provides a method of generating a T cell comprising a chimeric antigen receptor (CAR), the method comprising
(a) administering to a subject an agent that induces migration of stem cells from the subject's bone marrow into the subject's peripheral blood, thereby generating mobilized peripheral blood in the subject,
(b) obtaining mobilized peripheral blood from the subject,
(c) obtaining a T cell from the mobilized peripheral blood obtained in step (b),
(d) introducing a nucleic acid encoding a chimeric antigen receptor to the T cell obtained in step (c).

In another aspect, the present disclosure provides a T cell comprising a chimeric antigen receptor (CAR), wherein the T cell is generated by any of the methods disclosed herein.

In some aspects, the present disclosure provides a pharmaceutical composition comprising a T cell generated by any of the methods disclosed herein; and a pharmaceutically acceptable carrier.

In other aspects, the present disclosure provides a method of stimulating a chimeric antigen receptor (CAR)-mediated immune response to a target in a subject in need thereof, the method comprising

administering to the subject an effective amount of an T cell comprising a nucleic acid encoding a chimeric antigen receptor (CAR), wherein the T cell is made by any of the methods disclosed herein.

In one aspect, the present disclosure provides a method of treating a condition in a subject in need thereof, the method comprising

administering to the subject an effective amount of an T cell comprising a nucleic acid encoding a chimeric antigen receptor (CAR), wherein the immune cell is made by any of the methods disclosed herein.

In another aspect, the present disclosure provides a method of generating cells for allogeneic cell therapy, the method comprising

administering to a donor an agent that induces migration of stem cells from the donor's bone marrow to the donor's peripheral blood, thereby generating mobilized peripheral blood in the donor;

obtaining a T cell from a mobilized peripheral blood sample from the donor, wherein the T cell is subsequently administered to a recipient in need thereof, and

obtaining a stem cell from the same mobilized peripheral blood sample from the donor, wherein the stem cell is subsequently administered to a recipient in need thereof.

In some aspects, the present disclosure provides a method of generating cells for autologous cell therapy, the method comprising

administering to a subject an agent that induces migration of stem cells from the subject's bone marrow to the donor's peripheral blood, thereby generating mobilized peripheral blood in the subject;

obtaining a T cell from a mobilized peripheral blood sample from the subject, wherein the T cell is subsequently administered to the subject; and

obtaining a stem cell from the same mobilized peripheral blood sample from the subject, wherein the stem cell is subsequently administered to the subject.

In other aspects, the present disclosure provides a method of treating a condition in a subject in need thereof, the method comprising

administering to the subject a T cell comprising a chimeric antigen receptor (CAR), wherein the T cell is generated by introducing a CAR into a T cell obtained from a mobilized peripheral blood sample, and

administering to the subject a stem cell, wherein the stem cell is obtained from the same mobilized peripheral blood sample.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1C are a set of plots showing studies comparing the anti-tumor effects of CART^{mob} versus CART^{SS} where CART^{mob} denotes CART cells made from G-CSF mobilized peripheral blood and CART^{SS} denotes CART cells made from steady-state conditions (i.e. no exposure to G-CSF). T cells were collected from the same human donor. In these experiments, tumor refers to an AML cell line (MOLM14) and the CAR target is CD33. FIG. 1A: Bioluminescence to tumor (BLI) shows kinetics of response in mice treated with CART^{mob} or CARTS. FIG. 1B: A trend is shown to improved tumor control with CART cells derived from mobilized peripheral blood. FIG. 1C: A trend to improved CART expansion in vivo after injection of CART^{mob} compared with CART^{SS} (statistically significant at only one timepoint, D20).

FIG. 2 is a schematic showing T-cell expansions performed on PBMC that were collected at steady state (ss), or enriched CD4/8 T-cells after G-CSF mobilization (mob) cells.

FIGS. 3A-3I are graphs showing results of expansion analysis of CAR T-cells from donors collected at steady state or mobilized cells. No differences were seen in T-cell expansion kinetics, though population doublings trended towards being better in ssCAR33; (C-E) No differences were seen in CAR33 expression levels, or CD4:CD8 composition. (E-F) The proportion of CD4+ central memory (CM) T-cells was higher in mobCAR33, as was the proportion of CD45RO+CD27+ in both CD4 and CD8 subsets. (G-I) Higher expression of LAG3 was observed in CD4 CART33 cells manufactured from steady state cells, though the absolute expression levels remain low at the end of manufacturing. No other differences were observed between the CART33 products. Gating: Initial gating for both T-cell subsets and exhaustion panel—cells->single cells forward scatter->single cells side scatter->live->CAR+->CD3+->CD8 vs CD4. T-cell subsets were gated by CCR7 vs CD4RA to determine naive/T stem cell memory, central memory, effector memory and terminal effector memory subsets. Additional markers shown were also gated down from CD4 or CD8 populations as shown above. Cell expansion data compared by one-way ANOVA with correction for multiple comparison, showing aggregate data from 4 donors for population doublings, and for 3 donors for mean cell volume. All other figures show combined data from 4 normal donor CART33 cells, with all other statistical analysis by unpaired two-tailed t-tests. CART33 effectors from 4 donors were batched, being stained, acquired and analysed concurrently. fL femtoliters, Tscm T stem cell memory; CM central memory; EM effector memory; EFF/EMRA terminal effector memory. P-values ns>0.05, *<0.05.

FIGS. 4A-4H are graphs showing CAR T-cell expansion kinetics by donor.

FIGS. 5A-5F are graphs showing results of evaluation of in vitro and in vivo function of mobCAR33. No difference in cytotoxicity was observed after 24-hour co-culture of a CD33+ cell line (Molm14) with ssCAR33 or mobCAR33. Untransduced (UTD) cells are included as a control for non-CAR mediated cell death, and (B) Molm14 target cells after CD33KO serve as a negative control for this assay. Degranulation showed a difference in Donor 1 only, with higher GM-CSF production in mobCAR33 (C, E). No differences in cytokine production were seen in Donor 2. Cytotoxicity data (A-B) as aggregated from 3 donors with technical replicates, with n=9 samples at each data point. Statistical analysis by one-way ANOVA with correction for multiple comparisons. Degranulation data (C-F) are from individual normal donors, performed with technical replicates. Gating is on CD3+/GFP− cells (C-D), or on CAR+ T-cells only (E-F). Statistical analysis by unpaired two-tailed t-tests between ssCAR33 and mobCAR33 cells. Control conditions for degranulation assay are shown in FIG. 6. BLT bioluminescence; UTD Untransduced T-cells.

FIGS. 6A-6J are graphs showing conditions for degranulation assays, with no significant differences between T cells derived from mobilized or steady state blood.

FIGS. 7A-7H are graphs showing (A) Experimental design for an in vivo experiment, including 2 different dose levels (standard dose of 5×10⁶ CAR+ cells per mouse, and a stress dose of 2.5×10⁶ CAR+ cells per mouse) with (B) survival to 100 days. (C-D) In vivo T-cell expansion at each dose level tended to be higher in mobCAR33, and (E) greater IFNγ secretion was seen by mobCAR33 at standard dosing only; (F) No differences in mouse body weight, as a measure of toxicity, were observed between conditions, and (G-H) No differences in disease burden as assessed by bioluminescence imaging.

FIGS. 8A-8B are graphs showing all-cause survival or leukemia-specific survival, respectively (ssmobCAR33-1).

FIG. 9A-9D are graphs showing in vivo cytokine production (ssmobCAR33-3).

FIG. 10A-10H are schematic and graphs showing in vivo stress dose replicate (ssmobCAR33-2).

FIG. 11 is a panel showing CAR-T samples and characterizations for Donors 1-4.

FIG. 12 is a panel showing 34 markers used in for mass cytometry-based phenotyping.

FIGS. 13A-13F are graphs showing characterization of CD8 T cell subsets for CD33-CAR-T cells.

FIGS. 14A-14E are graphs showing CCR7, CD45RA, and CD95 expression CD8 T cell subsets for CD33-CAR-T cells.

FIGS. 15A-15B are heat map charts showing surface marker expression on CD4 and CD8 T cells.

FIGS. 16A-16F are heat map charts showing exhaustion marker expression for CD8 T cell subsets for CD33-CAR-T cells.

FIGS. 17A-17D are heat map charts and graphs showing exhaustion marker expression comparison between CD4 and CD8 T for CD33-CAR-T cells.

FIGS. 18A-18B are schematic and graph showing bioluminescence CAR-T killing assay.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means, in accordance with the present invention, any self-antigen which is recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “autoimmune disease” as used herein is defined as a disorder that results from an autoimmune response. An autoimmune disease is the result of an inappropriate and excessive response to a self-antigen. Examples of autoimmune diseases include but are not limited to, Addison's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barre syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. In some aspects, CARs comprise single-chain variable fragments (scFv) derived from monoclonal antibodies. The specificity of CAR designs may be derived from ligands of receptors (e.g., peptides). In some embodiments, a CAR can target cancers by redirecting the specificity of a T cell expressing the CAR specific for tumor associated antigens.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

The term “CRISPR/CAS,” “clustered regularly interspaced short palindromic repeats system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage.

In the type I1 CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering and therapeutics.

The term “CRISPRi” refers to a CRISPR system for sequence specific gene repression or inhibition of gene expression, such as at the transcriptional level.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes or a portion thereof.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of cells. In one embodiment, the cells that are expanded ex vivo increase in number relative to the number originally present in the culture. In another embodiment, the cells that are expanded ex vivo increase in number relative to other cell types in the culture.

The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

The term “hematopoietic stem cell” or “HSC” refers to an undifferentiated hematopoietic cell that is capable of differentiating into all blood cell types, myeloid and lymphoid cells. The HSC may reside in the bone marrow or be found elsewhere e.g. peripheral blood.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockout” or “KO” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

As used herein, “mobilized peripheral blood” is peripheral blood in a subject after administering to the subject an agent that induces migration of stem cells (e.g., CD34+ stem cells) from the subject's bone marrow to the subject's circulating or peripheral blood. Examples of such an agent include granulocyte colony-stimulating factor (G-CSF), granulocyte macrophage colony stimulating factor (GM-CSF), and plerixafor (CXCR4 antagonist). Mobilized peripheral blood is enriched in stem cells compared to unmobilized peripheral blood.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “portion thereof” refers to a part of or a fragment of the whole.

The term “hematopoietic progenitor cell” refers to an undifferentiated hematopoietic cell capable of differentiating into at least one blood cell type to several blood cell types, but not all blood cells like a HSC. Examples of hematopoietic progenitor cells include, but are not limited to, a common myeloid progenitor cell, megakaryocyte-erythrocyte progenitor cell, granulocyte-macrophage progenitor cell, monocyte-dendritic progenitor cell, and a common lymphoid progenitor cell.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

“Substantially complementary,” as used herein, refers to sequences of nucleotides where a majority or all of the bases in the primer sequence are complementary, or one or more bases are non-complementary, or mismatched.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention described herein includes compositions and methods of generating cells (e.g., T cells or stem cells) for use in cell therapy. The invention is based, at least in part, on the discovery that chimeric antigen receptor (CAR) T cells made using T cells harvested from peripheral blood mononuclear cells (PBMC) from G-CSF mobilized peripheral blood exhibited similar or superior anti-tumor effects and proliferation compared to CAR T cells made using T cells harvested from unmobilized peripheral blood. As described elsewhere herein, preliminary evidence is provided showing that CAR-T cells produced from peripheral blood mononuclear cells (PBMCs) harvested after mobilization with granulocyte colony stimulating factor (G-CSF) show better tumor control and proliferation in vivo than CAR-T cells manufactured using unstimulated PBMCs.

Certain embodiments of the invention comprise generating T cells to be genetically modified. In a particular embodiment, the T cells are to be genetically modified into chimeric antigen receptor (CAR) T cells. Some embodiments further comprise expanding the cells. Expansion may be prior to the step of introducing the nucleic acid. The cells may be cryopreserved then thawed prior to introducing the nucleic acids. The nucleic acid (e.g., the nucleic acid encoding a CAR) may be introduced by transducing the cell, or transfecting the cell, or electroporating the cell.

The invention also includes a modified cell that is generated according to the methods described herein. A pharmaceutical composition comprising the modified cell and a pharmaceutically acceptable carrier generated according to the methods described herein are also included in the invention.

Certain embodiments of the invention comprise methods for generating cells for autologous or allogeneic cell therapy, where immune cells and stem cells to be used for the therapy are harvested from a single mobilized blood sample (e.g. a single apheresis). Manufacture of CAR T-cells (and potentially other cell therapies) from G-CSF mobilized PBMC would (1) spare allogeneic donors undergoing G-CSF for stem cell harvest having 2 apheresis collections, with the attendant cost and risk, and (2) introduce a new source of more effective T cells into standard autologous CART cell therapy. Use of G-CSF mobilized PBMC that were previously cryopreserved for clinical purposes (as is often done for myeloma patients) could also improve the logistics and cost effectiveness of some CART cell therapies. For example, G-CSF stimulation is currently employed in collections for stem cell transplant, so in addition to the improved function described above, there may be efficiencies gained for patients receiving both stem cell transplant and cell therapy such that only a single G-CSF mobilized apheresis would be needed for both therapies rather than two separate collections. This applies for example, for CD33 CAR/CD33− HSC transplant therapy and CAR-T technology for Non-Hodgkin Lymphomas or multiple myeloma where autologous stem cell transplants have been part of standard-of-care for many years.

### Sources of T Cells

In one aspect, a method of generating a T cell comprising a chimeric antigen receptor (CAR) from mobilized peripheral blood is provided. In some embodiments, the method includes the step of administering to a subject an agent that induces migration of stem cells from the subject's bone marrow to the subject's peripheral blood. In some embodiments, the method includes the step of introducing a CAR into a T cell from the mobilized peripheral blood obtained from the subject. In some embodiments, the T cell is from peripheral blood mononuclear cells (PBMC) in the mobilized peripheral blood. In some embodiments, the PBMC is obtained from the subject via apheresis.

Mobilized peripheral blood can be generated by injecting a mobilizing agent to a subject (e.g., a donor or a patient). The mobilizing agent induces stem cells (e.g., CD34+ stem cells) to migrate from the bone marrow to circulating or peripheral blood. Mobilizing agents include, without limitation, G-CSF, GM-CSF, plerixafor (CXCR4 antagonist), and any combination thereof. In some embodiments, the mobilizing agent is G-CSF. In some embodiments, the mobilizing agent is recombinant human G-CSF. In some embodiments, the subject is administered with the mobilizing agent for at least 1, 2, 3, 4, or 5 days. In some embodiments, the subject is administered with the mobilizing agent for 1, 2, 3, 4, or 5 days. The mobilized peripheral blood provides a source of various cells, including immune cells and stem cells (e.g., CD34+ stem cells).

Illustrative cell immune cell types that can be obtained from the mobilized peripheral blood include T cells, B cells, dendritic cells, and other cells of the immune system. In one embodiment, the immune cell is a T cell. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation.

In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase.

### Activation and Expansion of T Cells

Whether prior to or after genetic modification of the T cells (e.g., to express a chimeric immune receptor), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof, and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle: cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

T cells that have been exposed to varied stimulation times may exhibit different characteristics. For example, typical blood or apheresed peripheral blood mononuclear cell products have a helper T cell population (TH, CD4*) that is greater than the cytotoxic or suppressor T cell population (Tc, CD8*). E vivo expansion of T cells by stimulating CD3 and CD28 receptors produces a population of T cells that prior to about days 8-9 consists predominately of TH cells, while after about days 8-9, the population of T cells comprises an increasingly greater population of Tc cells. Accordingly, depending on the purpose of treatment, infusing a subject with a T cell population comprising predominately of TH cells may be advantageous. Similarly, if an antigen-specific subset of Tc cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Sources of Stem Cells

In one aspect, methods for autologous or allogeneic cell therapy are provided, where immune cells and stem cells to be used for the therapy are harvested from a single mobilized blood sample (e.g. a single apheresis). Mobilized peripheral blood can be generated by injecting a mobilizing agent to a subject (e.g., a donor or a patient). The mobilizing agent mobilizes stem cells (e.g., CD34+ stem cells) from the bone marrow into circulating or peripheral blood. Mobilizing agents include, without limitation, G-CSF, GM-CSF, plerixafor, and any combination thereof. In some embodiments, the mobilizing agent is G-CSF. In some embodiments, the subject is administered with the mobilizing agent for at least 1, 2, 3, 4, or 5 days. In some embodiments, the subject is administered with the mobilizing agent for 1, 2, 3, 4, or 5 days. The mobilized peripheral blood provides a source of various cells, including immune cells (e.g., T cells) and stem cells (e.g., CD34+ stem cells).

In one embodiment, stem cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, the cells are isolated from peripheral blood by lysing the red blood cells and depleting the monocytes, for example, by centrifugation through a PERCOLL™ gradient. In any event, a specific subpopulation of HSC or progenitor cells can be further isolated by positive or negative selection techniques.

Enrichment of a cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD34+ cells by negative selection, a monoclonal antibody cocktail typically includes antibodies to CD4, CD5, CD8, CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

The cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In one embodiment, the stem cell is obtained from cells selected from mobilized peripheral blood. In another embodiment, the cell is CD34+.

### Expansion of Stem Cells

The present invention includes a population of stem cells comprising the modified cell described herein. In one embodiment, the method for generating the modified cell described herein also includes expanding the cell or the modified cell. In one embodiment, the expansion is prior to the step of introducing the nucleic acid. In yet another embodiment, the expansion is prior to the step of introducing the nucleic acid. In some embodiments, the cells disclosed herein can be expanded by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the cells are expanded in the range of about 20 fold to about 50 fold.

The cells can be incubated in cell medium in a culture apparatus for a period of time or until the cells reach high cell density for optimal passage before passing the cells to another culture apparatus. The culturing apparatus can be of any culture apparatus commonly used for culturing cells in vitro. The cell medium may be replaced during the culture of the cells at any time. Preferably, the cell medium is replaced about every 2 to 3 days. The cells are then harvested from the culture apparatus whereupon the cells can be used immediately or cryopreserved to be stored for use at a later time. In one embodiment, the invention includes cryopreserving the expanded cells. The cryopreserved cells are thawed prior to introducing nucleic acids into the cell.

In another embodiment, the method further comprises isolating the cell and expanding the cell. In another embodiment, the invention further comprises cryopreserving the cell prior to expansion. In yet another embodiment, the invention further comprises thawing the cryopreserved cell prior to introducing the nucleic acids.

The culturing step as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for HSC or progenitor cell culture include an appropriate media (e.g., Minimal Essential Media or RPMI Media 1640 or, X-vivo 15, (Lonza)) that may contain factors necessary for proliferation and viability, such as but not limited to, serum (e.g., fetal bovine or human serum), GM-CSF, insulin, IFN-gamma, interleukin-1 (IL-1), IL-3, IL-4, IL-6, IL-7, IL-10, IL-12, IL-15, SCF, TGF-beta, TNF-α and TPO. or any other additives for the growth of cells known to the skilled artisan. In one embodiment, the cell culture includes IL-3, IL-6, GM-CSF, SCF and TPO. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of HSC or progenitor cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02).

The medium used to culture the cells may include an agent that can stimulate the modified cells to expand. The cell modified by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the modified cell expands in the range of about 20 fold to about 50 fold, or more by culturing the modified cell.

### Genetic Modification of Immune Cells or Stem Cells

The immune cells and/or stem cells obtained from mobilized peripheral blood of the invention can be further genetically modified prior to administration to a subject. The immune cell (e.g., T cell) may be modified, for example, into a chimeric antigen receptor (CAR) immune cell by introducing a nucleic acid encoding a CAR into the immune cell. The CAR T cell may be further modified to delete genes and/or overexpress genes to enhance the CAR T cell's function. Likewise, the stem cell may be genetically modified prior to administration to a subject.

In one embodiment, the stem cell is genetically modified to delete an endogenous gene. In one embodiment, the stem cell is genetically modified to decrease or abolish expression of CD33.

In one embodiment, the immune cell is a T cell. In one embodiment, the T cell is genetically modified to a T cell comprising a CAR. In one embodiment, the CAR comprises an intracellular signaling domain, a transmembrane domain, and an antigen binding domain. In another embodiment, the CAR further comprises an intracellular domain of a costimulatory molecule. In one embodiment, the antigen binding domain specifically binds a tumor antigen. In another embodiment, the tumor antigen is CD33. In another embodiment, the tumor antigen is CD19, CD20, CD22, CD38, CD123 or BCMA.

### CRISPR/Cas

In some embodiments, the immune cell and/or the stem cells obtained from mobilized peripheral blood is modified prior to administration to a subject using the CRISPR/Cas system. Genome editing using programmable nucleases enables precise editing at specific genomic loci, which can be used to remove deleterious mutations or insert protective mutations. To date, there are three major classes of nucleases—zinc finger nucleases (ZFNs), transcription activator-like effector nucleases (TALENs), and clustered, regularly interspaced, short palindromic repeat (CRISPR)-associated nucleases. Of these, CRISPR-associated nucleases have proven to be markedly superior to the others in terms of the ease and simplicity of use.

The CRISPR/Cas system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The Cas9 protein, under direction from the gRNA, binds to its target DNA sequence and cuts both strands of the DNA at a specific locus. This double-stranded DNA break is repaired by either non-homologous end joining (NHEJ) or homology-directed repair (HDR). NHEJ frequently causes small insertions or deletions (indels) at the breakage site that can lead to a frameshift mutation of the protein encoded by the gene. HDR utilizes a repair template that is copied into the gene, thus engineering specific mutations.

The CRISPR/CAS system can thereby be engineered to cleave virtually any DNA sequence by redesigning the gRNA in cell lines (such as 293T cells), primary cells, CAR T cells, and stem and progenitor cells. In one aspect, the invention includes a modified hematopoietic stem or progenitor cell comprising a nucleic acid capable of decreasing expression of an endogenous gene or a portion thereof, wherein the endogenous gene encodes a polypeptide comprising an antigen domain targeted by a chimeric antigen receptor (CAR).

One example of a CRISPR/Cas system used to inhibit gene expression, CRISPRi, is described in U.S. Publication No.: 2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. The CRISPR/CAS system can also simultaneously target multiple genomic loci by co-expressing a single CAS9 protein with two or more gRNAs, making this system uniquely suited for multiple gene editing or synergistic activation of target genes. In one aspect, a modified hematopoietic stem or progenitor cell is generated by introducing a nucleic acid capable of decreasing expression of an endogenous gene or a portion thereof into the cell, wherein the endogenous gene encodes a polypeptide comprising an antigen domain targeted by a chimeric antigen receptor (CAR). In such an embodiment, the nucleic acid capable of decreasing expression of the endogenous gene or a portion thereof is a CRISPR system. In some embodiments, the CRISPR system includes a Cas expression vector and a guide nucleic acid sequence specific for the endogenous gene. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

In one embodiment, introducing the CRISPR system comprises introducing an inducible CRISPR system. The CRISPR system may be induced by exposing the hematopoietic stem or progenitor cell to an agent that activates an inducible promoter in the CRISPR system, such as the Cas expression vector. In such an embodiment, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

The guide nucleic acid sequence is specific for a gene and targets that gene for Cas endonuclease-induced double strand breaks. The sequence of the guide nucleic acid sequence may be within a locus of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

The guide nucleic acid sequence may be specific for any gene, such as an endogenous gene that would reduce immunogenicity or reduce sensitivity to a CART therapy. The endogenous gene of the present invention encodes a polypeptide comprising an antigen domain targeted by a CAR. In one embodiment, the guide nucleic acid sequence is specific for the endogenous gene that encodes a tumor antigen. In yet another embodiment, the guide nucleic acid sequence is specific for the endogenous gene that encodes CD33 or CD123.

The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

### CARs

In one aspect, a method of making an immune cell (e.g., a T cell) comprising a chimeric antigen receptor (CAR) is provided. In some embodiments, the method contains the step of introducing a nucleic acid encoding a chimeric antigen receptor (CAR) into an immune cell, where the immune cell is obtained from mobilized peripheral blood from a subject, where the mobilized peripheral blood is generated by administering to the subject an agent that mobilizes stem cells from the subject's bone marrow into the subject's peripheral blood. In another aspect, a method of making an immune cell comprising a chimeric antigen receptor (CAR), where the method contains the steps of (a) administering to a subject an agent that mobilizes stem cells from the subject's bone marrow into the subject's peripheral blood, thereby generating mobilized peripheral blood in the subject, (b) obtaining mobilized peripheral blood from the subject, (c) obtaining an immune cell from the mobilized peripheral blood obtained in step (b), (d) introducing a nucleic acid encoding a chimeric antigen receptor to the immune cell obtained in step (c). In other aspects, the invention provides immune cells (e.g., T cells) made by the methods described herein.

CARs are typically used as a therapy in adoptive cell transfer. The CAR is an artificial receptor expressed on a T cell that is engineered to specifically bind to an antigen and activate the T cell as an immune effector cell. In many instances, the antigen targeted by the CART cells is an endogenous gene that is expressed on normal and diseased cells. Thus, the CART cells target both normal and diseased cells for elimination.

Antigen Binding Domain

A CAR usually includes an extracellular domain that comprises an antigen binding domain. In some embodiments, the antigen binding domain of the CAR specifically binds to the antigen on a target cell. In other embodiments, the antigen binding domain of the CAR specifically binds to a tumor antigen.

Examples of antigens targeted by the CAR of the invention includes CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTi); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member IA (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTi); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECl2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

Transmembrane Domain

With respect to the transmembrane domain, the CAR immune cells (e.g., CAR T cells) the present invention can be designed to comprise a transmembrane domain that is fused to the extracellular domain (e.g., antigen binding domain). In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the receptor. A glycine-serine doublet provides a particularly suitable linker.

Cytoplasmic Domain

The cytoplasmic domain or otherwise the intracellular signaling domain of the chimeric antigen receptor (CAR) expressed by immune cells (e.g., T cells) of the invention is responsible for activation of at least one of the normal effector functions of the immune cell in which the chimeric antigen receptor has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein that transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the receptor of the invention comprises a cytoplasmic signaling sequence derived from CD3 zeta.

In a preferred embodiment, the cytoplasmic domain of the receptor can be designed to comprise the CD3-zeta signaling domain by itself or combined with any other desired cytoplasmic domain(s) useful in the context of the receptor of the invention. For example, the cytoplasmic domain of the receptor can comprise a CD3 zeta chain portion and a costimulatory signaling region. The costimulatory signaling region refers to a portion of the receptor comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD3, CD7, LIGHT, NKG2C, B7-H3, and a ligand that specifically binds with CD83, and the like. Thus, while the invention is exemplified primarily with CD28 and 4-1BB as the co-stimulatory signaling element, other costimulatory elements are within the scope of the invention.

The cytoplasmic signaling sequences within the cytoplasmic signaling portion of the receptor of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signaling domain of CD3-zeta. In another embodiment, the cytoplasmic domain is designed to comprise any combination of CD3-zeta, CD28, 4-1BB, and the like.

### Introduction of Nucleic Acids

Methods of introducing a nucleic acid into the immune cell or stem cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce the nucleic acid into the cell, a variety of assays may be performed to confirm the presence of the nucleic acid in the cell. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one aspect, the invention includes a method for generating a T cell comprising a chimeric antigen receptor (CAR) comprising introducing a nucleic acid encoding a CAR into a T cell obtained from a mobilized peripheral blood sample from a subject. In another embodiment, the invention includes a method of generating a stem cell from the same sample of mobilized peripheral blood. In one embodiment, the method comprises further modifying the CAR T cell (e.g., introducing genetic modifications that enhance, for example, the function or proliferation of the CAR T cell). In one embodiment, the method further comprises modifying the stem cell. In one embodiment, the stem cell is modified to decrease expression of an endogenous gene in the cell, wherein the endogenous gene encodes a polypeptide comprising an antigen domain to be targeted by a CAR. In another embodiment, the stem cell is modified to introduce a modified endogenous gene into the cell, wherein the modified endogenous gene encodes a modified polypeptide lacking the antigen domain targeted by the CAR. In such an embodiment, one nucleic acid may be introduced using the same or a different method from that used to introduce the modified endogenous gene into the cell.

RNA

In one embodiment, the nucleic acid introduced into the cell comprises a RNA. In another embodiment, at least one component of the CRISPR system comprises RNA. In yet another embodiment, the guide nucleic acid sequence is a RNA. In another embodiment, the RNA comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

PCR can be used to generate a template for in vitro transcription of RNA which is then introduced into cells. Methods for performing PCR are well known in the art. Primers for use in PCR are designed to have regions that are substantially complementary to regions of the DNA to be used as a template for the PCR. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the template. Alternatively, UTR sequences that are not endogenous for the template can be added by incorporating the UTR sequences into the forward and reverse primers or by any other modifications of the template. The use of UTR sequences that are not endogenous to the template can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the RNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

The RNAs described herein may be introduced into the cell by a variety of methods known in the art. In some embodiments, the RNA is electroporated into the cells. In one embodiment, the CRISPR system comprises a RNA that is electroporated into the cells. In yet another embodiment, the CRISPR system comprises at least one guide nucleic acid sequence that is a RNA and electroporated into the cells.

The disclosed methods can be applied to the modulation of cell activity in order to provide therapy to the subject in the fields of cancer, acute and chronic infections, and autoimmune diseases. The disclosed methods can involve targeting stem cells, and also can include methods for assessing the ability of the genetically modified cell to kill a target cancer cell.

The methods also provide the ability to control the level of expression over a wide range by changing, for example, the promoter or the amount of input RNA, making it possible to individually regulate the expression level.

One advantage of RNA transfection methods of the invention is that RNA transfection is essentially transient and a vector-free. A RNA transgene can be delivered to a cell and expressed therein, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of the cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Therapy

The immune cells and/or stem cells described herein may be included in a composition for therapy. The composition may include a pharmaceutical composition and further include a pharmaceutically acceptable carrier. A therapeutically effective amount of the pharmaceutical composition comprising the cells may be administered.

In one aspect, a method of stimulating a chimeric antigen receptor (CAR)-mediated immune response to a target in a subject in need thereof is provided. In another aspect, an immune cell for use in a method of stimulating a chimeric antigen receptor (CAR)-mediated immune response to a target in a subject in need thereof is provided. In another aspect, a method of treating a condition in a subject in need thereof is provided. In still another aspect, an immune cell for use in a method of treating a condition in a subject in need thereof is provided. In another aspect, a method of increasing in vivo proliferation of a CAR immune cell (e.g., a CAR T cell) is provided. In some embodiments, the methods include the step of administering to the subject an effective amount of an immune cell containing a nucleic acid encoding a chimeric antigen receptor (CAR), where the immune cell is made by the methods described herein (e.g., CAR immune cell is made using an immune cell that was obtained from mobilized peripheral blood). In some embodiments, the target is a tumor. In some embodiments, the condition is cancer. In some embodiments, the immune cell is a T cell.

In another aspect, a method of generating cells for autologous cell therapy is provided. In yet another aspect, a method of generating cells for allogeneic cell therapy is provided. In some embodiments, the methods include the step of administering to a subject (e.g. a donor or patient) an agent that mobilizes stem cells from the subject's bone marrow into the donor's peripheral blood, thereby generating mobilized peripheral blood in the subject; obtaining an immune cell from a mobilized peripheral blood sample from the subject, wherein the immune cell is to be administered to a subject (e.g., a recipient or the same patient); and obtaining a stem cell from the same mobilized peripheral blood sample from the subject, wherein the stem cell is to be administered to the subject (e.g., a recipient or the same patient). In another aspect, the invention includes a method of treating a condition in a subject in need thereof, where the method includes administering to the subject an immune cell (e.g., a T cell) comprising a chimeric antigen receptor (CAR) and administering to the subject a stem cell, where the immune cell is generated by introducing a CAR into a T cell obtained from a mobilized peripheral blood sample, and the stem cell is obtained from the same mobilized peripheral blood sample.

In certain embodiments, T cells are harvested from the mobilized peripheral blood sample and further modified into CD33 CAR T cells, and stem cells are obtained from the same mobilized peripheral blood sample and the stem cells are further modified to decrease or delete CD33 expression. The CD33 CAR T cells and CD33 knockout stem cells can be administered to a subject in need thereof.

The cells described herein can be administered to a subject, preferably a mammal, even more preferably a human. In one embodiment, the modified cell differentiates into at least one blood cell type in the subject. In another embodiment, the modified cell is capable of self-renewal after administration into the subject.

In one embodiment, the condition is a cancer. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In certain embodiments, the cancer is breast cancer, prostate cancer, ovarian cancer, cervical cancer, skin cancer, pancreatic cancer, colorectal cancer, renal cancer, liver cancer, brain cancer, lymphoma, leukemia, or lung cancer. In certain embodiments, the cancer is a leukemia, such as acute myeloid leukemia.

Further, the modified cells can be administered to a subject, preferably a mammal, even more preferably a human, to suppress an immune reaction. The modified cells can be administered to suppress an immune reaction, such as those common to autoimmune diseases such as diabetes, psoriasis, rheumatoid arthritis, multiple sclerosis, GVHD, enhancing allograft tolerance induction, transplant rejection, and the like. In addition, the cells of the present invention can be used for the treatment of any condition in which a diminished or otherwise inhibited immune response, especially a cell-mediated immune response, is desirable to treat or alleviate the disease.

Further, the modified cells can be administered to a subject, preferably a mammal, even more preferably a human, to treat a condition, such as an autoimmune disease. Examples of various autoimmune diseases include but are not limited to Examples of autoimmune disease include but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

The cells generated as described herein can also be modified and used to treat inflammatory disorders. Examples of inflammatory disorders include but are not limited to, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

In another embodiment, the modified cell described herein may be used for the manufacture of a medicament for the treatment of an immune response in a subject in need thereof.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous, allogeneic or xenogeneic with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

### Pharmaceutical Compositions

Pharmaceutical compositions of the present invention may comprise the cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the modified cells described herein may be administered at a dosage of 10⁴ to 10⁹ cells/kg body weight, preferably 10⁵ to 10⁶ cells/kg body weight, including all integer values within those ranges. Cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments of the present invention, the cells are expanded and modified using the methods described herein, or other methods known in the art where the cells are expanded to therapeutic levels, and administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the modified cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludarabine, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Green, M. R. & Sambrook, J., Cold Spring Harbor Laboratory Press, 2012); “Oligonucleotide Synthesis, a practical approach” (Paselk R. A., edited by Gait, M. J., IRL Press, Oxford, 1984); “Culture of Animal Cells: A Manual of Basic Technique and Specialized Applications, Sixth Edition” (Freshney, R. I., John Wiley & Sons, Inc., 2010); “Methods in Enzymology” (Vol. 152, Guide to Molecular Cloning Techniques, Berger and Kimmel, Eds., San Diego: Academic Press, Inc., 1987); “Handbook of Experimental Immunology” (Herzenberg L. A., Weir, D. M., Blackwell, C., Wiley, 1997); “Gene Transfer Vectors for Mammalian Cells” (Miller and Calos, eds., Cold Spring Harbor Laboratory, Cold Spring Harbor, 1987); “Short Protocols in Molecular Biology” (Ausubel, F. M., et al., ed., John Wiley & Sons, 2002); “Polymerase Chain Reaction: Principles, Applications and Troubleshooting” (Babar, M. E., publisher VDM Verlag Dr. Müller, 2011); “Current Protocols in Immunology” (Colligan, J. E., et al., ed., Greene Pub. Associates and Wiley-Interscience, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in these experiments are now described.

### Example 1

### CAR T Cells Manufactured from G-CSF Mobilized PBMC

The attributes of T cells obtained from steady-state compared with G-CSF mobilized blood were evaluated. PBMC from normal volunteers before and after administration of 4-5 days of G-CSF. 10-20 cc of blood was collected through venipuncture at baseline. Three (3) to five (5) L of apheresed PBMC was collected after G-CSF injection.

This study sought to determine whether CAR T cells could be manufactured from G-CSF mobilized PBMC. In this study, the null hypothesis was that G-CSF primed T cells (henceforth, CART^{mob}) would proliferate less well ex vivo and would mediate a poorer anti-tumor effect than CART cells made from steady-state unmobilized T cells from the same donor (henceforth, CART^{SS}). If the null hypothesis was rejected (that is, the anti-tumor function of CART^{mob}=antitumor function of CART^{SS}) this would open the way to using a single G-CSF mobilized apheresis as the source for both stem cells and T cells for clinical studies that either (1) rely on allogeneic T cells for patients who have or will undergo a standard-of-care allogeneic stem cell transplant, or (2) are designed as a tandem gene-edited stem cell transplant plus CAR T cell administration protocol. The need for only a single apheresis session is expected to improve logistics, expedite time to infusion, reduce costs, and potentially increase the pool of potential healthy T cell donors (since current clinical practice is to consent unrelated donors only for G-CSF mobilized apheresis, and a separate overture must be made to the same donors when/if an unmobilized apheresis is desired, for the purpose of standard-of-care donor lymphocyte infusion for the treatment of relapse post-transplant).

The results of the experiments are now described.

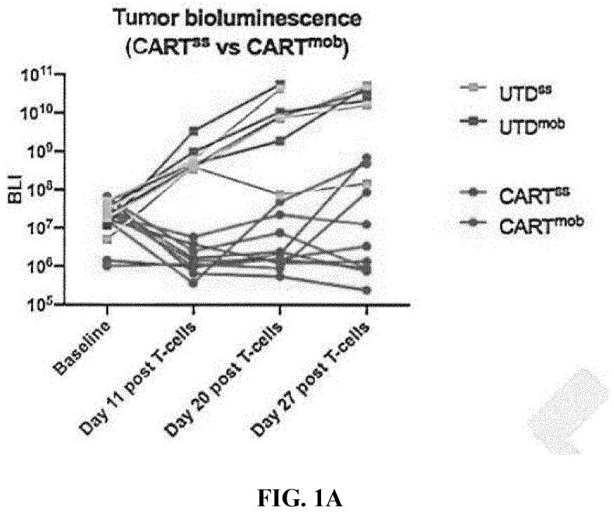

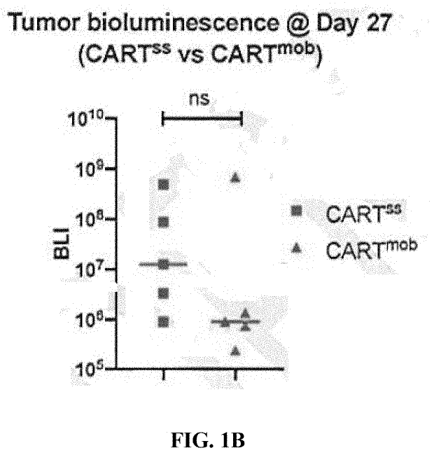

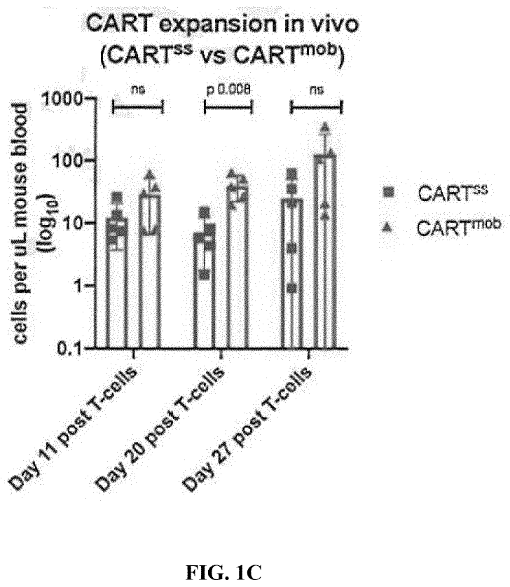

The in vitro studies revealed a broadly similar anti-tumor effect when CART^{mob} were compared to CART^{SS}. Surprisingly, in vivo xenograft studies showed that the anti-tumor effect and proliferation of CART^{mob} may be superior to those of CART^{SS} (FIGS. 1A-1C), without evidence of increased toxicity to the xenografted mice. Further studies are being pursued to verify these results and to explain the mechanism for the differential efficacy.

### Example 2

### CAR T-Cell Manufacture and Analysis of ssCAR33 and mobCAR33 Infusion Product

### Methods

### Collection and Cryopreservation of Paired Normal Donor Cells for CAR T-Cell Manufacture

Healthy volunteer donors underwent a blood draw of 30 mL of whole blood anti-coagulated in ethylenediaminetetraacetic acid (EDTA). Peripheral blood mononuclear cells (PBMC) were isolated by Ficoll gradient separation and cryopreserved in 90% fetal bovine serum (FBS) with 10% dimethylsulfoxide (DMSO), in a single vial per donor, in liquid nitrogen. After 5 days of subcutaneous G-CSF therapy at 10 μg/kg/day, the donor underwent a single apheresis (10-15 L, depending on estimated blood volume), and the mobilized apheresis product was collected in accordance with standard clinical procedures in the clinical apheresis unit at The University of Pennsylvania. The apheresis product was resuspended in autologous plasma to a total volume of 500 mL and transported fresh to the Product Development Laboratory for processing. CD34+ enrichment by positive selection was performed using the FDA approved CliniMACS CD34 reagent system (Miltenyi Biotech) according to the manufacturer's protocol. CD34+ cells were diverted to a specific research project, while the CD34− fraction was rested overnight at 4° C. in the Miltenyi negative fraction collection buffer. Prior to cryopreservation in liquid nitrogen, CD4/8 enrichment was performed using CD4 and CD8 magnetic microbeads (Miltenyi, CD4 beads 130-045-101, CD8 beads 130-045-201) to deplete myeloid cells by positive selection of T-cells. Cells were cryopreserved in aliquots of maximum 50×10⁶ cells per vial in 90% FBS with 10% DMSO and stored in liquid nitrogen. All donors were managed in accordance with an IRB approved protocol at The University of Pennsylvania for this research.

### CAR Constructs

The CAR33 construct used in this work has been previously described (Gill S, Tasian S K, Ruella M, et al., Preclinical targeting of human acute myeloid leukemia and myeloablation using chimeric antigen receptor-modified T cells, Blood, 123(15):2343-2354 (2014); Kenderian S S, Ruella M, Shestova O, et al., CD33-specific chimeric antigen receptor T cells exhibit potent preclinical activity against human acute myeloid leukemia. Leukemia, 29(8):1637-1647 (2015); Kim M Y, Yu K R, Kenderian S S, et al., Genetic Inactivation of CD33 in Hematopoietic Stem Cells to Enable CAR T Cell Immunotherapy for Acute Myeloid Leukemia, Cell, 173(6):1439-1453.e1419 (2018)). In brief, the CAR33 construct used is a a second generation CAR with a 41BB co-stimulatory domain and an single chain variable fragment (scFv) that is based on the anti-CD33 monoclonal antibody gemtuzumab (Baron J, Wang E S, Gemtuzumab ozogamicin for the treatment of acute myeloid leukemia. Expert Rev Clin Pharmacol. 2018; 11(6):549-559; Appelbaum F R, Bernstein I D, Gemtuzumab ozogamicin for acute myeloid leukemia, Blood, 130(22):2373-2376) (2017)). Cloning was performed in-house and research-grade lentivirus was used for the CART33 cells in this work, with manufacture as described below.

### Lentiviral Activation and Titration

Human embryonic kidney (HEK-293T) cells were purchased from ATCC (CRL-3216) and were propagated in Dulbecco's Modified Eagle Medium (DMEM, Gibco, 11594486) at 37° C. and 5% CO2. Early passage cells (fewer than 5 passages) were then cryopreserved in 90% FBS with 10% DMSO and stored in liquid nitrogen. The 293T producer cells were later thawed and plated at 5×10⁴ cells/cm² in DMEM in a 175 cm² flask (Corning, CLS431080) and incubated at 37° C. and 5% CO₂. When at 80% confluency, cells were transfected using 90 μL per flask of Lipofectamine 2000 (ThermoFisher, 11668019) with the relevant CAR transgene combined with lentiviral packaging plasmids at the following ratios; transgene 15 μg; gag-pol 18 μg; rev-tat 18 μg; vsv-g 7 μg. Cell supernatant was collected at 24- and 48-hours post transfection, vacuum filtered (cellulose acetate membrane, pore size 0.45 μm, membrane diameter 70 mm, Corning, CLS430512) and then concentrated using an ultra-centrifuge (2.5 hours at 25,000 g), and stored at −80 degrees Celsius until required. Lentivirus was titrated functionally using primary human T-cells 24-hours after activation with CD3/CD28 microbeads (Dynabeads, ThermoFisher, 40203D, donated by the University of Pennsylvania Cell and Vaccine Production Facility) in accordance with our institutional protocol. CAR expression was determined by flow cytometry 72-86 hours after the addition of lentivirus, using a recombinant CD33-APC conjugate manufactured in-house, as described below.

### T-Cell Activation and CAR T-Cell Expansion

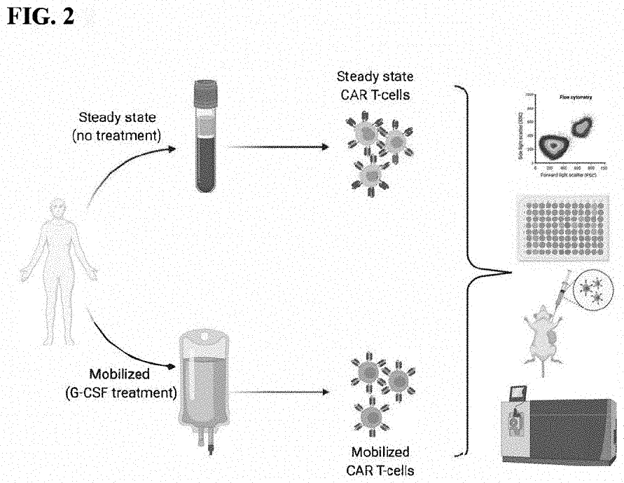

T-cell expansions were performed on PBMC that were collected at steady state (ss), or enriched CD4/8 T-cells after G-CSF mobilization (mob) cells, as described above (FIG. 2). For each donor, CAR T-cells using both ss and mob starting cells were manufactured in parallel, and an untransduced (UTD) T-cell expansion was also performed for each starting cell product. Cryopreserved donor cells were thawed, washed in sterile room temperature phosphate buffered saline (PBS) and incubated with an RNA and DNA targeting endonuclease (Benzonase™, Millipore Sigma, E1014-5KU) for 30 minutes at 37° C. Cells were then resuspended in an in-house T-cell media (X-VIVO-10 Serum-free Hematopoietic Cell Medium (Lonza, 04-380Q), with 5% Human AB Serum (Gemini, 100-512), 1% GlutaMAX (ThermoFisher 25050061) and 1% penicillin/streptomycin. Cells were plated at a density of approximately 2×10⁶/mL and rested overnight at 37° C. with 5% CO2. The following morning, cells were counted and re-suspended at 1×10⁶/mL in T-cell media, which was supplemented with 1L7 and IL15 (5 ng/mL per cytokine, PeproTech 200-07 & 200-15 respectively). CD3/CD28 beads (Dynabeads®, ThermoFisher, 40203D, donated by The University of Pennsylvania Cell and Vaccine Production Facility) were washed and combined with PBMC or T-cells at a ratio of three beads per cell. Cells were then expanded in accordance with our institution's T-cell expansion protocol, de-beaded using a magnet, and subsequently cryopreserved when the mean cell volume (MCV) dropped below 300 fL, unless otherwise stated.

### CAR T-Cell Phenotyping by Flow Cytometry

CAR quantification on CART33 cells was achieved by staining with a recombinant human CD33 protein (r-CD33, Sino Biological, 12238-H05H) conjugated in-house with an APC fluorophore using a conjugation kit (Abcam, ab201807). The reagent (rCD33-APC) was then titrated using previously manufactured CART33 cells, and the dilution yielding the optimum signal:noise ratio was selected for subsequent staining.

Additional phenotyping was performed on CAR T-cells by flow cytometry; cells were washed in PBS and incubated with the relevant antibody at room temperature protected from UV-light for 20 minutes. Staining for CCR7, when performed, was incubated at 37° C. for 45 minutes as a single stain, after which time additional antibodies were added (Table 1) and cells were incubated at room temperature protected from UV light for a further 20 minutes. Cells were washed and resuspended in 300 μL PBS and filtered with a 70 μm filter prior to acquisition on a BD Fortessa. Data analysis was performed using FlowJo™, v10.6.1.

TABLE 1
CAR T-cell phenotyping reagents.

| | T-cell memory subsets | T-cell activation/exhaustion |
|---|---|---|
| | Target protein (clone) | Target protein (clone) |
| | Manufacturer; product TD | Manufacturer; product TD |
| FITC | CD4 (OKT4) | LAG3 (11C3C65) |
| | Invitrogen; 11-0048-42 | BioLegend; 369308 |
| PE | CD57 (HCD57) | CD39 (Al) |
| | BioLegend; 322312 | BioLegend; 328208 |
| PE/C y7 | CCR7 (G043H7) | CD38 (HIT2) |
| | BioLegend; 353226 | eBioscience; 25-0389-42 |
| APC | CAR33 | CAR33 |
| | Sino rhCD33-fc/AbCam APC | Sino rhCD33-fc/AbCam APC |
| PerCPCy5.5 | CXCR3 (G025H7) | TIM3 (F38-2E2) |
| | BioLegend 353714 | BioLegend; 345016 |
| APC/Cy7 | CD3 (SP34-2) | CD3 (SP34-2) |
| | BB Pharminogen; 557757 | BB Pharminogen; 557757 |
| BV421 | CD95 (DX2) | CD8a (RPA-T8) |
| | BD Bioscience; 562616 | BioLegend; 301036 |
| LD Aqua | viability | viability |
| BV711 | CD8a (RPA-T8) | PD1 (EH12.2H7) |
| | BioLegend; 301044 | BioLegend; 329928) |
| BV570 | CD45RO (UCHL1) | CD4 (RPA-T4) |
| | BioLegend; 304225 | BioLegend; 300534 |
| AF700 | CD27 (0323) | TIGIT |
| | BioLegend; 302814 | R&D FAB7898N |
| BV786 | CD45RA (H1100) | VISTA (MIH65) |
| | BD Biosciences; 563870 | BD Bioscience; 749640 |

Generation of Molm14 CD33 Knock-Out Cells with CRISPR for In Vitro Assays

The guide RNA (gRNA) used for CD33 knock-out (GUCAGUGACGGUACAGGA, was developed in our lab and previously described (Kim M Y, Yu K R, Kenderian S S, et al., Genetic Inactivation of CD33 in Hematopoietic Stem Cells to Enable CAR T Cell Immunotherapy for Acute Myeloid Leukemia, Cell, 173(6):1439-1453.e1419 (2018)), was manufactured using the TranscriptAid T7 High Yield Transcription Kit (ThermoFisher, K0441) from a linearised plasmid cloned in-house. Molm14 cells (ATCC, CRL-1582) were resuspended in SF Cell line buffer kit in 20 μL format (Lonza kit V4XC-2032). Cells were electroporated using the Lonza-41D nucleofector with pulse code EO-100, in combination with 5 μg of TruCut V2 Cas9 protein (ThermoFisher, A36498) and 2.5 μg gRNA per 1×10⁶ Molm14 cells. The gRNA and Cas9 protein were complexed for 10 minutes at room-temperature to form a ribonuclear protein (RNP) prior to addition to the cell suspension. Post-electroporation, cells were maintained at a density of 5×10⁶/mL overnight, in accordance with the manufacturers protocol, before being expanded in culture for analysis. After 7 days, CD33 surface expression was evaluated by flow cytometry. After confirmation of knock-out, cells were sorted three times using a BD FACS Melody to obtain a cell population with <0.01% residual CD33 by flow cytometry. CD33-negative cells were then expanded and cryopreserved in 90a FBS with 10% DMSO, and stored in liquid nitrogen for subsequent use. A CD33KO-Molm14 cell line with CBG/GFP expression was subsequently manufactured by transduction of abicistronic plasmid containing both transgenes, and sorted by flow cytometry for GFP+ cells three times using a BD FACS Melody, and cryopreserved as described above.

### CAR T-Cell In Vitro Degranulation/Cytokine Production Assay

CAR T-cells (or UTD T-cells) were first stained for CAR33, using the rCD33-APC conjugate as described previously. T-cells were then co-cultured for 4-hours with either media only, media containing T-cell mitogens (phorbol myristate acetate and ionomycin, PMA/IO, BioLegend, 423302) or GFP+ Molm14 target cells (CD33 wild-type or CD33KO, as described above). Cells were also co-cultured with anti-CD28 & CD49d activating antibodies, Monensin (1000×, BioLegend, 420701) and a surface CD107a staining reagent (Table 2).

TABLE 2
CAR T-cell reagents for in vitro and in vivo assays.

| | Alternative | | | | Catalogue |
|---|---|---|---|---|---|
| Antigen | names | Clone | Conjugate | Supplier | number |
| Human | Human | n/a | n/a | BioLegend | 422302 |
| Fc | TruStain FcX | | | | |
| block | | | | | |
| Mouse Fc | TruStain FcX | n/a | n/a | BioLegend | 101320 |
| block | anti-mouse | | | | |
| | CD16/32 | | | | |
| Viability | Live/dead | n/a | n/a | ThermoFisher | L34957 |
| dye | Aqua Cell | | | | |
| | stain kit | | | | |
| CD3 | CD3e | OKT3 | APC | BioLegend | 317318 |
| CD33 | SIGLEC-3 | HIM3-4 | APC | eBioscience | 14-0339-82 |
| CD33 | SIGLEC-3 | P67.6 | FITC | BioLegend | 303304 |
| CD45 | Leukocyte | 2D1 | BV421 | BioLegend | 368521 |
| (human) | common | | | | |
| | antigen | | | | |
| | (human) | | | | |
| CD45 | Leukocyte | 30-F11 | APC-Cy7 | BioLegend | 103116 |
| (murine) | common | | | | |
| | antigen | | | | |
| | (murine) | | | | |
| CD107a | Lysosome- | LAMP-1 | PE-Cy7 | BioLegend | 328618 |
| | associated | | | | |
| | membrane | | | | |
| | glycoprotein 1 | | | | |
| IFNγ | Interferon | B27 | PE | BioLegend | 506507 |
| | gamma | | | | |
| IL2 | Interleukin 2 | 5344.111 | PECF594 | BD | 562384 |
| | | | | Biosciences | |
| GM-CSF | Granulocyte- | BVD2- | BV421 | BioLegend | 562930 |
| | macrophage | 21C11 | | | |
| | colony- | | | | |
| | stimulating | | | | |
| | factor | | | | |
| TNFα | Tumor | MAb11 | AF700 | BD | 502928 |
| | necrosis factor | | | Biosciences | |
| | alpha | | | | |

After incubation for 4-hours at 37° C. with 5% CO₂, cells were washed and stained for CD3 and with a viability dye (LIVE/DEAD Fixable Aqua Dead Cell Stain Kit, ThermoFisher, L34957) and incubated for 15 minutes at room temperature, protected from UV light. Cells were then fixed and permeabilised (FIX and PERM, ThermoFisher, GAS004) according to the manufacturer's protocol, and intra-cellular staining performed for IFNγ, TNFα, IL2 and GM-CSF, (Table 2). After a further 20-minute incubation at room temperature and protected from UV light, cells were washed and re-suspended in PBS, and CAR T-cells analysed by flow cytometry, gating on live, GFP-negative, CD3+ cells, and additionally on CAR+ cells where indicated in the figure legend. Events were acquired using a BD Fortessa (three laser), and data analysis was performed using FlowJo™, v10.6.1. Cell quantification where relevant was performed using CountBright Absolute Counting Beads for flow cytometry (ThermoFisher, C36950).

### CAR T-Cell In Vitro Killing Assay

T-cells (UTD or CAR expressing) were plated at a starting concentration of 5×10⁶/mL in a 96-well flat-bottom black well plate, and serial dilutions into T-cell media performed to achieve a total volume of 100 uL per well. Wells thus contained a maximum of 5×10⁵ UTD or CART cells, with halving of cell numbers in each row. Click beetle green luciferase/green fluorescent protein (CBG/GFP) transduced Molm14 target cells (wild-type or CD33KO) as previously described, were washed and resuspended at 1×10⁶ cells per mL, and 100 uL of this suspension combined with the T-cells, generating a co-culture at a range of effector to target ratio of 5:1, 2:5:1, 1.25:1 etc. The plate was incubated for 24 hours at 37° C. with 5% CO2, luciferin was added (D-Luciferin potassium salt, GoldBio, LUCK-100) and cells resuspended by pipetting, and bioluminescence (BLT) captured using a plate reader. Specific lysis was calculated by the equation [(BLT targets only well−BLT test well)/BLT targets only well]×100=percent specific cell lysis of target by effector.

### Murine Studies

Mice used were bred in-house from stock originally purchased from The Jackson Laboratory. Mice were given irradiated chow and water ad libitum, and housed in BSL-2 cages as per our TACUC protocol. NOD^{-scid} TL2Rgamma^{null} (NSG strain, stock number 005557) were injected via tail vein with 1×10⁶ tumour cells, with no conditioning given prior to cell injection. Engraftment of click beetle green luciferase expressing tumour cell lines was confirmed by bioluminescence (defined as >1.0×10⁶ photons per second, p/s), after intra-peritoneal injection of 150 uL of luciferin (D-Luciferin potassium salt, GoldBio, LUCK-100) and imaged using a Xenogen TVTS-200 Spectrum camera (captured bioluminescence using automated acquisition setting). Tmaging data was analysed using Living Tmage version 4.4 (Caliper LifeSciences, PerkinElmer). Effector T-cells were thawed, washed in sterile PBS, strained though a 70 uM filter and resuspended in sterile heparinised PBS, at a concentration of 5×10⁶ CAR+ cells/mL. Prior to injection, CAR T-cell products were normalized for CAR T-cell expression by dilution with untransduced cells (UTD) from the same donor manufactured in the same expansion, resulting in the injection of the same total number of T-cells per mouse between conditions, at the same concentration of CAR+ cells. Whole blood was drawn via retroorbital bleeds following T-cell injection, and separated into plasma and PMBC for down-stream analysis. PBMC were analysed by flow cytometry, after red cell lysis and Fc blocking against both mouse and human Fc receptors. Mice were sacrificed at TACUC protocol-defined end points, or at the termination of the study, whichever came first, and where relevant bone marrow mononuclear cells and/or other haematopoietic or non-haematopoietic tissues were harvested and preserved for subsequent analysis. Mice to be used in rechallenge experiments were imaged for tumor burden as outlined above, and mice with BLT less than 1.0×10⁶ p/s were considered to be in remission from leukemia. These mice, along with sex matched controls, were injected via tail-vein injection with 1×10⁶ Molm14 CBG/GFP+ tumor cells per mouse, and analysis of tumor growth monitored by BLT.

### Cytokine Analysis of Murine Plasma

The plasma fraction from blood obtained from mice by retro-orbital bleed was separated by centrifugation at 1000 g for 10 minutes, removed by pipette and cryopreserved at −80 degrees. Plasma samples from all living mice at each time point per in vivo study were batched and subsequently analysed using the BD Cytometric Bead Array (CBA) Human Th1/Th2/Th17 Cytokine Kit (BD Biosciences, 560484) in accordance with the manufacturer's instructions, and events acquired on using a BD Fortessa (three laser). Data was then analysed using BD FCAP Array software (v3.0.1).

### Statistical Analysis and Figures

Analysis was performed using PRISM GraphPad Version 8.0.1 (145). P-values unless otherwise stated indicate; ns>0.05, *<0.05; **<0.01; ***<0.001, ****<0.000. Graphics were created with Biorender.com, and the relevant statistical method used for each figure are described in the figure legends.

### Results

CAR T-Cell Manufacture and Analysis of ssCAR33 and mobCAR33 Infusion Product

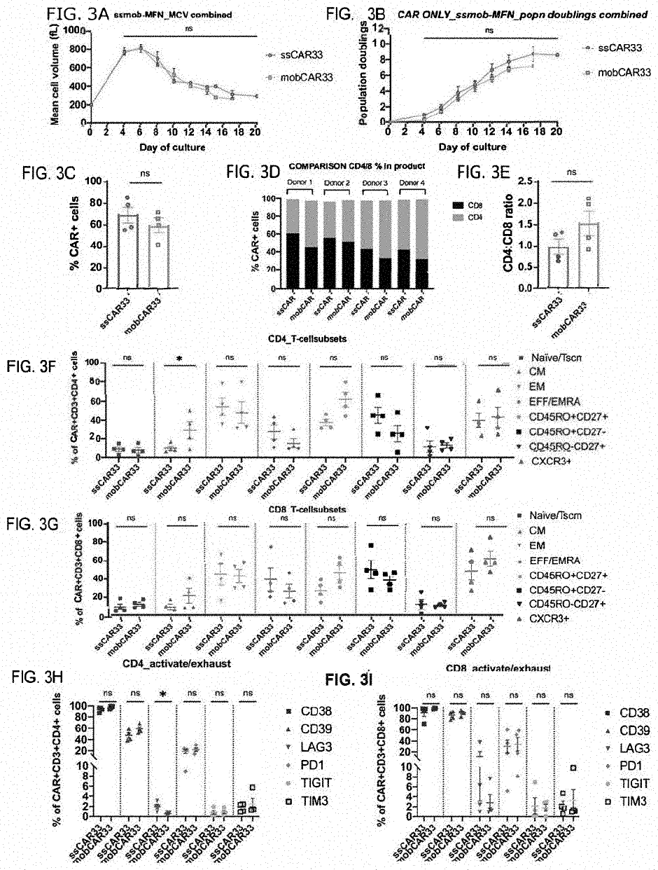

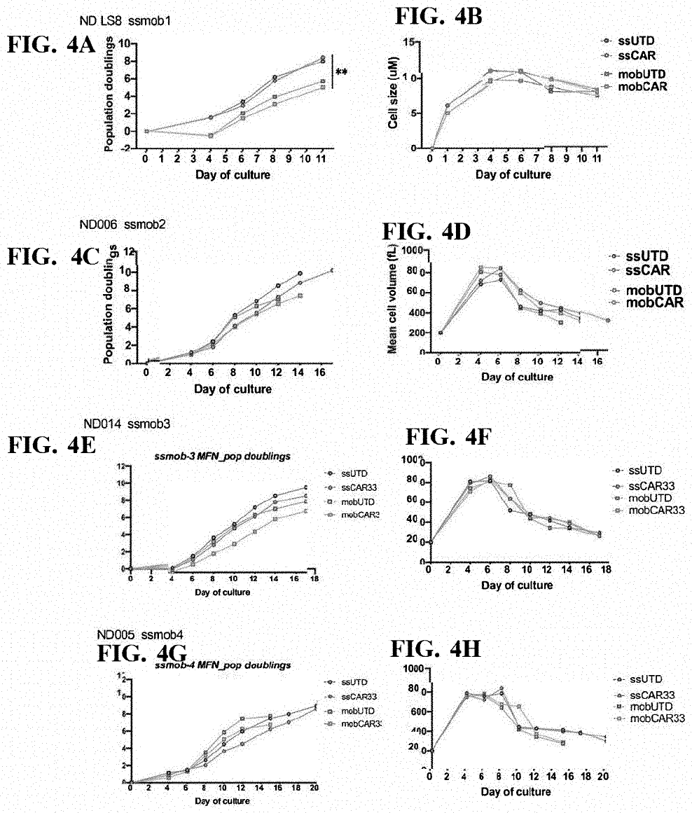

CAR T-cell expansions were made from paired samples from 4 normal donors, using either peripheral blood mononuclear cells (PMBC) collected at steady state (ss) or CD4/CD8 enriched PBMC after 5 days of G-CSF treatment (mob). Expansion kinetics were similar between CART33 cells made from steady state cells (ssCART33) or from mobilized cells (mobCART33), FIGS. 3A-B and while there was a trend towards lower CAR expression and a higher CD4:8 ratio in mobCART33, these differences were not statistically significant, FIG. 3C-E. Additional phenotyping of the CART33 products were performed by flow cytometry, with focus on T-cell memory subsets (Gattinoni L, Speiser D E, Lichterfeld M, Bonini C., T memory stem cells in health and disease, Nat Med, 23(1):18-27 (2017)) and T-cell activation/exhaustion markers expressed at the end of manufacturing. The proportion of CD4+ central memory (CM) T-cells was higher in mobCART33, as was the proportion of CD45RO+CD27+ in both CD4+ and CD8+ subsets. Higher expression of LAG3 was observed in CD4+ ssCART33 compared with mobCART33, though the absolute expression levels were low. No other differences were observed between CART33 cells manufactured from steady state or mobilized cells.

Evaluation of In Vitro and In Vivo Function of mobCAR33

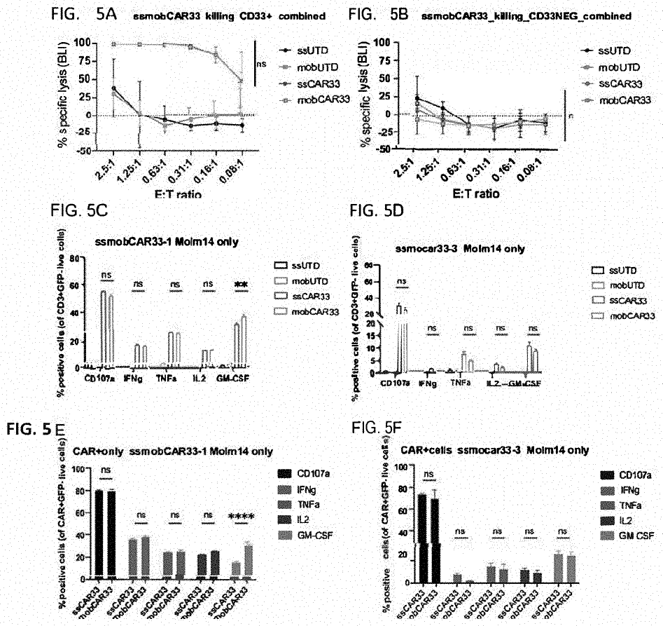

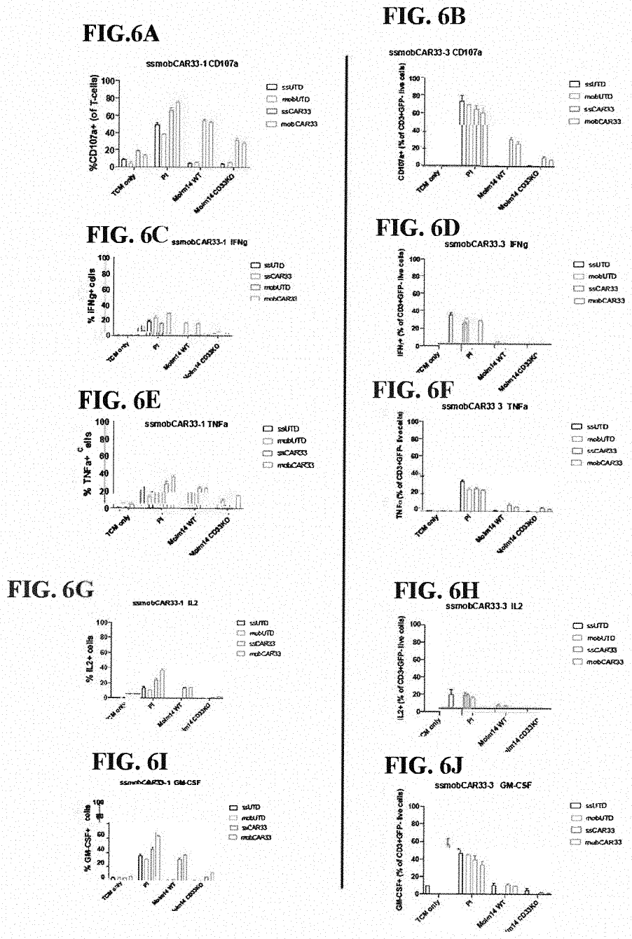

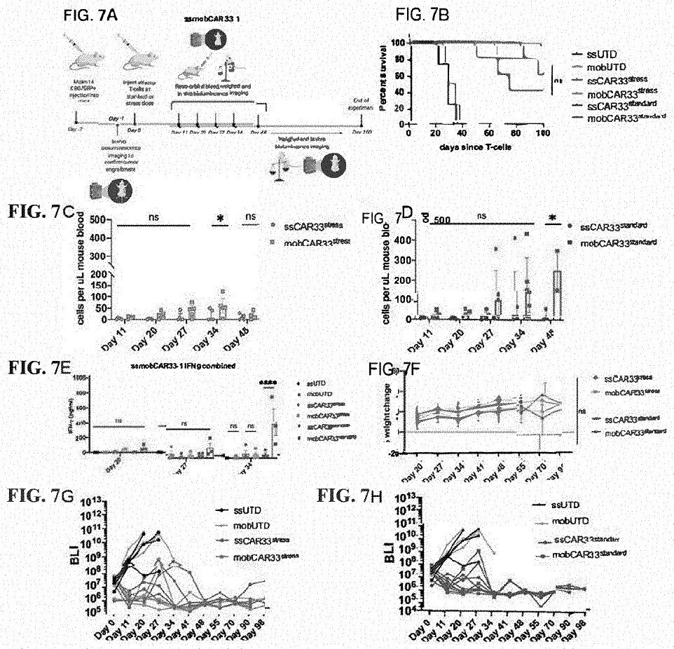

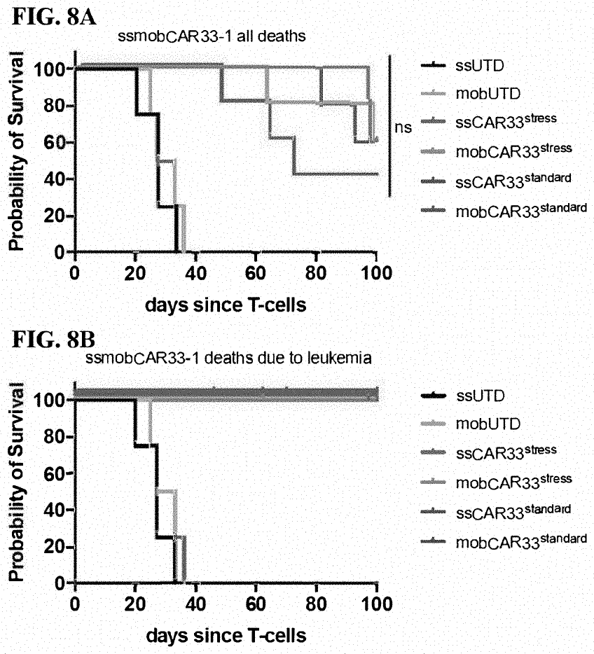

No differences in specific lysis of a CD33+ AML cell line (Molm14) by mobCART33 compared to ssCART33 were observed (FIG. 5A) nor in in vitro cytokine production by CART33 after co-culture with Molm14 cells (FIG. 5C-E), other than greater GM-CSF production by mobCART33 in one donor only. Based on these in vitro findings, an in vivo experiment was performed to assess the anti-leukemic potency of mobCART33 compared with ssCART33 in an NSG mouse model, after engraftment with CD33+ Molm14 cells. After tumor engraftment was confirmed by BLT, mice were treated with either standard dosing for this model (5×10⁶ CAR+ cells per mouse, which is expected to cure most animals) or at a stress dose (2.5×10⁶ CAR+ cells per mouse, which will control disease but usually not cure animals thus allowing evaluation of potentially subtle differences between effector T-cells). CAR T-cell expansion was equal at most time points, with enhanced expansion of mobCART33 demonstrated at later time points at both standard and stress dose levels (FIG. 5C-D). Enhanced TFNα production in plasma at Day 34 was produced by mobCART33 at the standard dose only (FIG. 5E) though no differences in body weight were observed to suggest significant cytokine release syndrome (CRS) was present (FIG. 7F). Changes in disease burden by BLT indicate that the majority of mice in this experiment were cured (FIG. 7G-H), which is typically seen at the standard dose in this model, though would not be expected at the stress dose, suggesting that this particular donor's cells (Donor #1) may have exhibited higher than usual levels of xenoreactivity. While no differences in survival were seen (FIG. 7B), more early deaths occurred in the standard dose treated animals and subsequent analysis indicated that these mice did not have evidence of leukemia by BLT at the time of their death (FIG. 8). This suggests that the cause of death may have been related to toxicity of the T-cells manifesting as graft versus host disease (GvHD), which was observed in many animals treated with the standard dose (both ssCART33 and mobCART33).

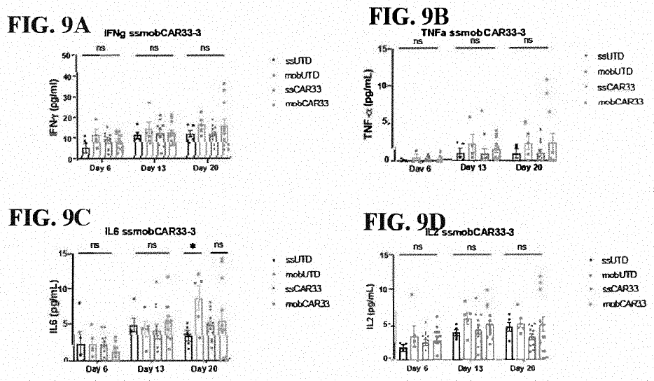

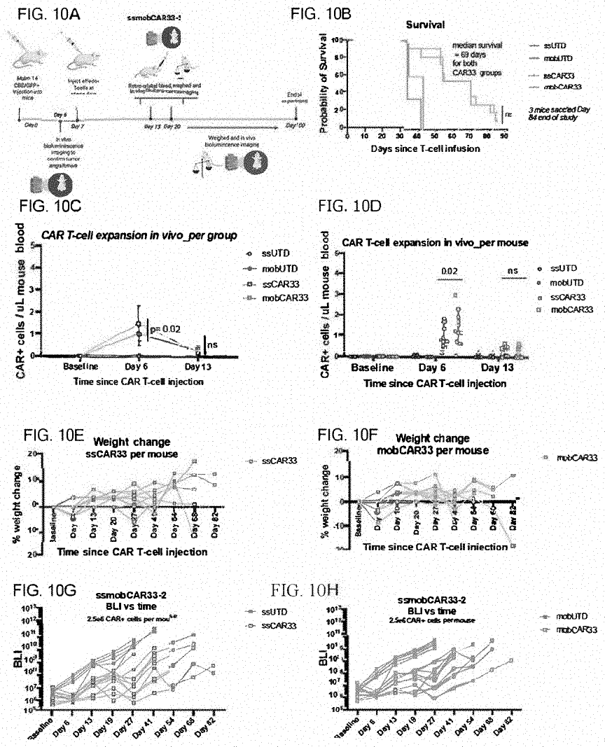

To confirm the equivalence in terms of anti-leukemic potency of mobCART33 with ssCART33 observed in these experiments, a replicate in vivo experiment was performed using the stress dose (2.5×10⁶ CAR+ cells per mouse) only from a different normal donor (FIG. 10A). No significant differences were seen in survival, cytokine production (FIG. 9) or body weight, and while only very small CART33 expansion in vivo was observed, this was slightly higher in the mobCART33 treated mice, compared with ssCART33 (p=0.02, (FIG. 10C-D). This experiment was terminated early (Day 84) as all mice had clear evidence of progressive disease.

### Additional T-Cell Phenotyping by Time-of-Flight Mass Cytometry (CyTOF)

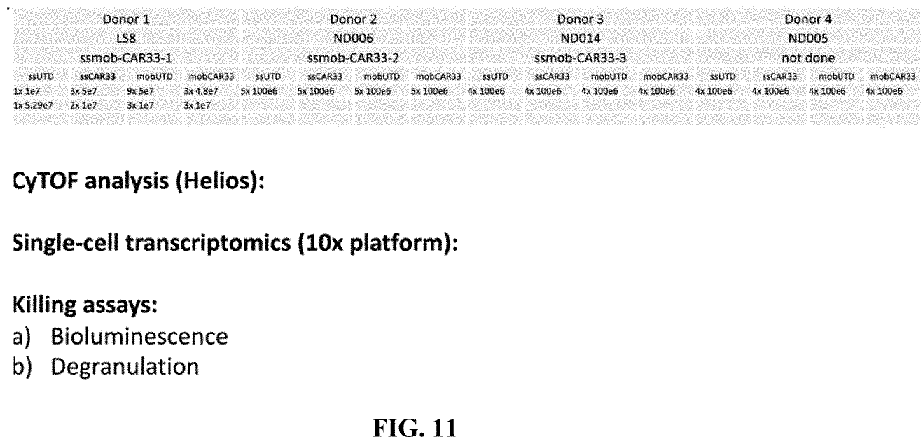

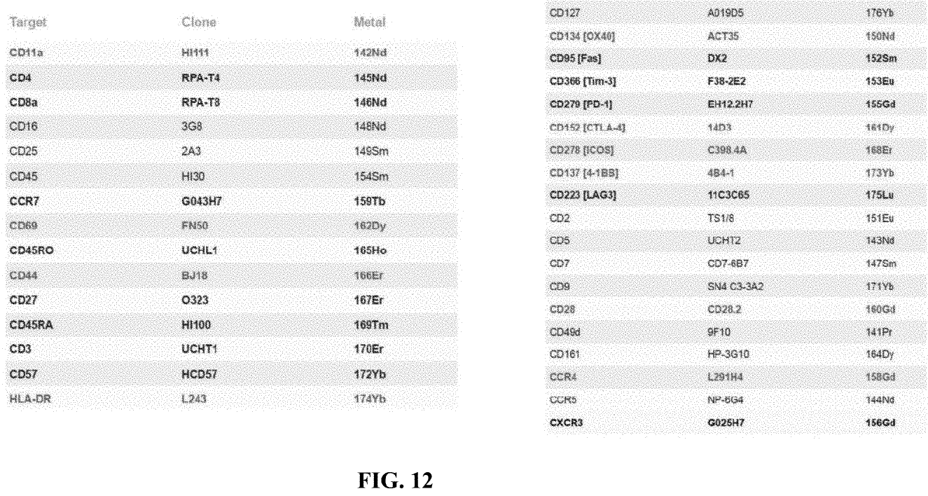

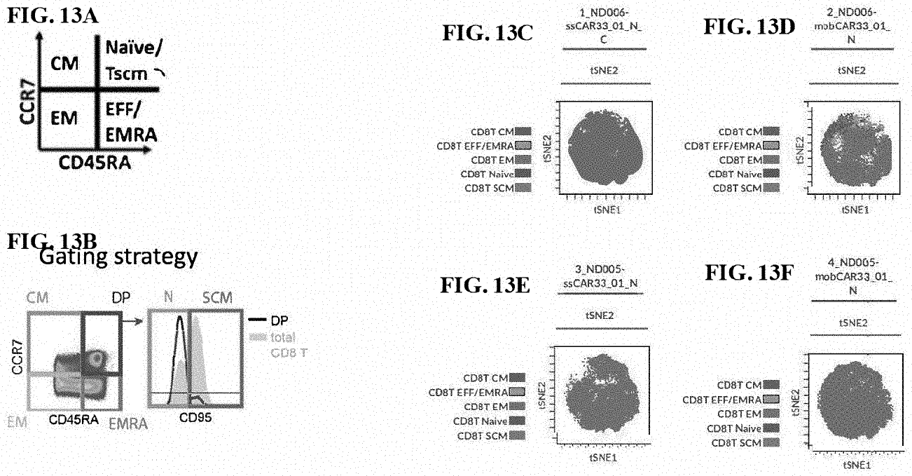

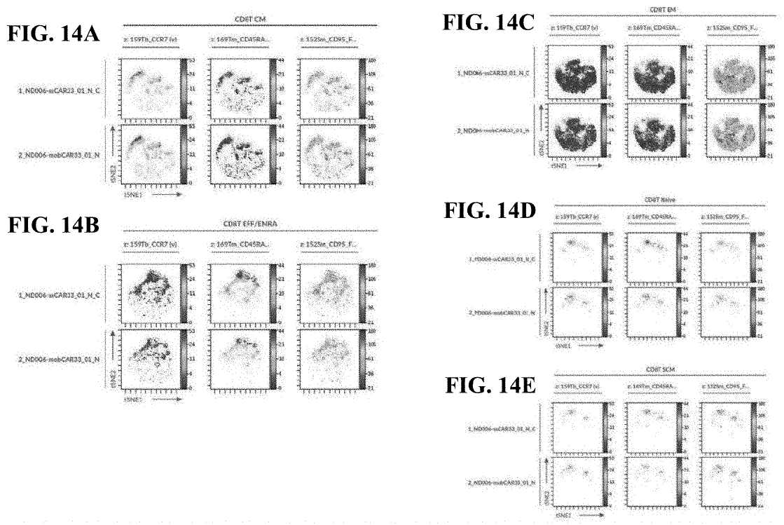

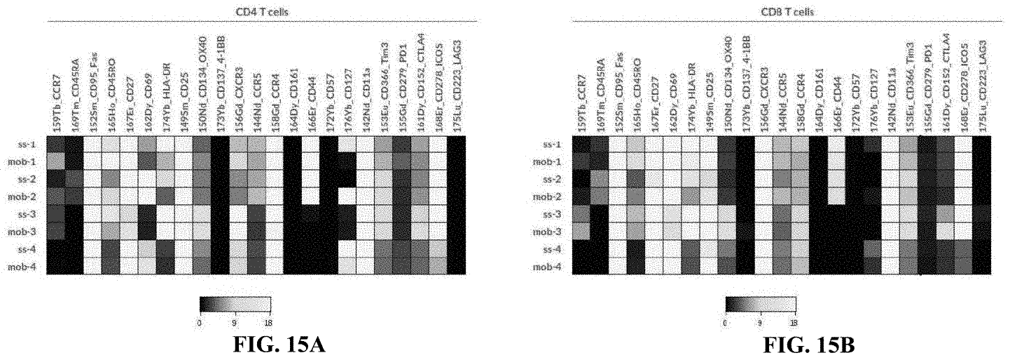

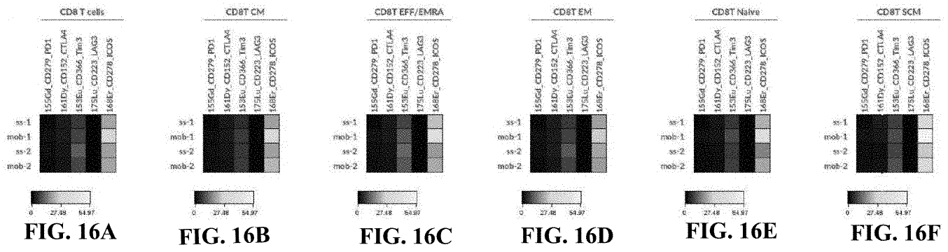

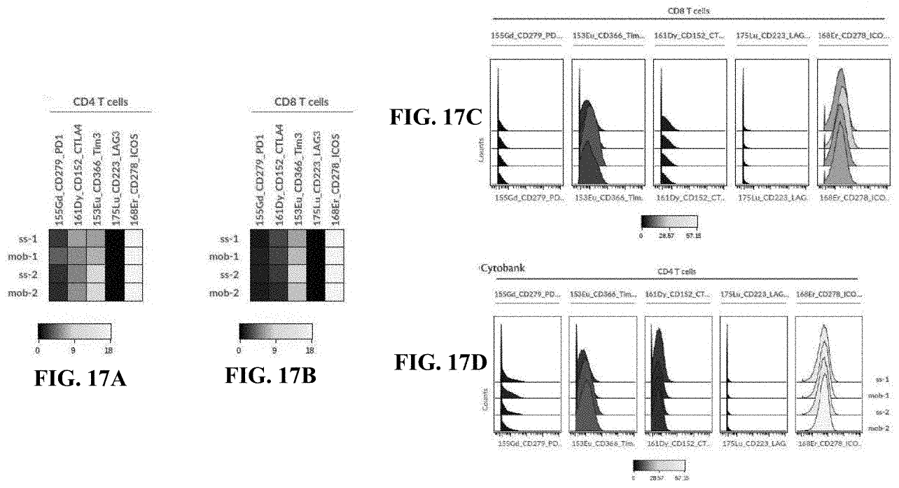

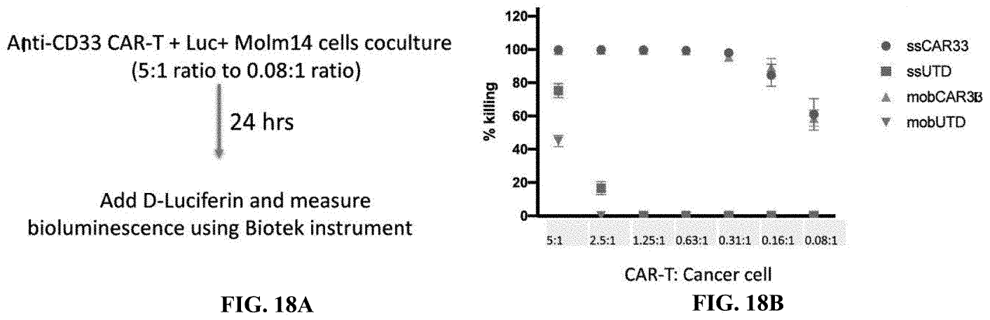

CAR-T samples and characterization are shown in FIG. 11. A 34 marker panel (FIG. 12) shows no obvious differences between 2 donors that have been tested to date show no differences in T-cell memory subsets in the CAR product in mobCART33 with ssCART33 (FIGS. 13-17). A replicate killing assay using the same effector and target cells showed almost identical results (FIG. 18, compare with FIG. 2A).

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of generating a T cell comprising a chimeric antigen receptor (CAR) from mobilized peripheral blood, the method comprising administering to a subject an agent that induces migration of stem cells from the subject's bone marrow to the subject's peripheral blood and introducing a CAR into a T cell from the mobilized peripheral blood obtained from the subject.

2. The method of claim 1, wherein the T cell is from peripheral blood mononuclear cells (PBMC) obtained from the subject via apheresis.

3. The method of claim 2, wherein the PBMC is obtained from the subject 1, 2, 3, 4, or 5 days after administration of the agent.

4. The method of claim 1, wherein the agent is G-CSF.

5. The method of claim 1, wherein the CAR comprises an antigen binding domain, an intracellular signaling domain, and a transmembrane domain.

6. The method of claim 5, wherein the CAR further comprises an intracellular domain of a costimulatory molecule.

7. The method of claim 5, wherein the antigen binding domain specifically binds a tumor antigen.

8. A method of generating a T cell comprising a chimeric antigen receptor (CAR) from mobilized peripheral blood, the method comprising
administering to a subject an agent that induces migration of stem cells from the subject's bone marrow to the subject's peripheral blood, thereby generating mobilized peripheral blood in the subject, and
introducing a CAR into a T cell obtained from peripheral blood mononuclear cells (PBMC) in the mobilized peripheral blood obtained from the subject.

9. The method of claim 8, wherein the PBMC is obtained from the subject via apheresis.

10. The method of claim 8, wherein the PBMC is obtained from the subject 1, 2, 3, 4, or 5 days after administration of the agent.

11. The method of claim 8, wherein the agent is G-CSF.

12. The method of claim 8, wherein the CAR comprises an antigen binding domain, an intracellular signaling domain, and a transmembrane domain.

13. The method of claim 12, wherein the CAR further comprises an intracellular domain of a costimulatory molecule.

14. The method of claim 12, wherein the antigen binding domain specifically binds a tumor antigen.

15. A method of generating stem cells and T cells, the T cells comprising a chimeric antigen receptor (CAR), from a single source, the method comprising
administering to a subject an agent that induces migration of stem cells from the subject's bone marrow to the subject's peripheral blood, thereby generating mobilized peripheral blood in the subject,
introducing a CAR into a T cell obtained from a mobilized peripheral blood obtained from the subject, and
obtaining a stem cell from the same mobilized peripheral blood sample obtained from the subject.

16. The method of claim 15, wherein the T cell is from peripheral blood mononuclear cells (PBMC) obtained from the subject via apheresis.

17. The method of claim 16, wherein the PBMC is obtained from the subject 1, 2, 3, 4, or 5 days after administration of the agent.

18. The method of claim 15, wherein the agent is G-CSF.

19-30. (canceled)

31. A T cell comprising a chimeric antigen receptor (CAR), wherein the T cell is generated by the method of claim 1.

32. A pharmaceutical composition comprising the T cell of claim 31 and a pharmaceutically acceptable carrier.

33-70. (canceled)
